(19) United States
(12) Patent Application Publication
(10) Pub. No.: US 2024/0052274 A1
(43) Pub. Date: Feb. 15, 2024

(54) SOAP-INFUSED COMPRESSED SPONGE

(71) Applicant: Esther Herzog, Brooklyn, NY (US)

(72) Inventor: Esther Herzog, Brooklyn, NY (US)

(21) Appl. No.: 18/105,835

(22) Filed: Feb. 4, 2023

Related U.S. Application Data
(60) Provisional application No. 63/396,728, filed on Aug. 10, 2022.

(51) Int. Cl. C11D 17/04; C11D 11/00; A61Q 19/10; A61K 8/02

(52) U.S. Cl. CPC C11D 17/049; C11D 11/0023; A61Q 19/10; A61K 8/0216

(57) ABSTRACT

The present invention relates to a compressed sponge that is infused with a liquid bath soap and subsequently packaged for transport. In a preferred embodiment, the sponge is compressed to approximately five millimeters in height for packaging. Upon release from the packaging and placed in water, the invention expands to its original height.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### CROSS REFERENCE TO RELATED APPLICATION

This application claims the benefit of US Provisional Patent Application No. 63/396,728, filed Aug. 10, 2022, and incorporated herein by reference.

### FIELD

The present invention relates to portable sponges and liquid soaps for use by people in a hotel while traveling. It can also be conveniently used at home, or by the elderly and disabled.

### BACKGROUND OF THE INVENTION

Traditionally, people pack both their favorite liquid soap and bath sponge before embarking on travel. Once in their hotel room, they then put the liquid soap into the soap, as they would do at home. If their favorite soap is a bar, then must be brought in a case, which isn't disposable and is inconvenient.

However, packaging a full bottle of liquid uses a lot of room in one's suitcase or travel bag, plus its weight is an inconvenience.

The present invention is not described in the prior art.

### SUMMARY OF THE INVENTION

A bath hygiene device comprising: a sponge compressed to approximately five millimeters in height to a create compressed sponge; soap added to said compressed sponge;

wherein said compressed sponge and said soap are packaged together as a single unit in a plastics wrapper; and

wherein the height of said compressed sponge expands by approximately three times when removed some from said packaging.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 depicts the invention as packaged and sold, as viewed from above with invention laying flat.

FIG. 2 depicts a perspective view of the invention when fully compressed, as viewed from above with invention laying flat and removed from the packaging.

FIG. 3 depicts the invention in use. Note, it is no longer compressed, but expanded.

### DETAILED DESCRIPTION OF THE PREFERRED EMBODIMENTS

For a more complete understanding of the present invention, the objects and advantages thereof, reference is now made to the ensuing descriptions.

The present invention is a compressed sponge infused with liquid soap. Once removed from the packaging and no longer compressed, and placed under a stream of water, the invention gradually expands to a large sponge.

The present invention overcomes the difficulty of transporting a full bottle of liquid soap along with a sponge while traveling. Instead, a person need only take the invention to enjoy the use of a sponge and liquid soap while traveling.

The invention is compressed, lightweight and disposable. These attributes make it very convenient for travel.

With a little bit of water, the invention becomes an all-in-one shower companion.

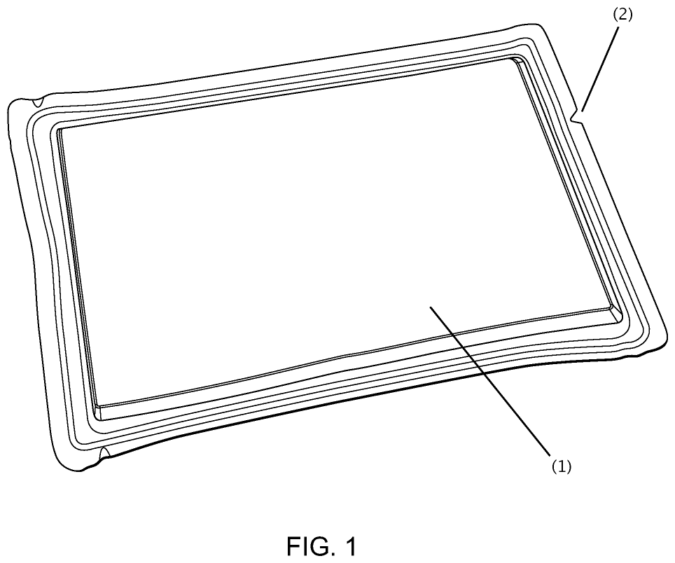

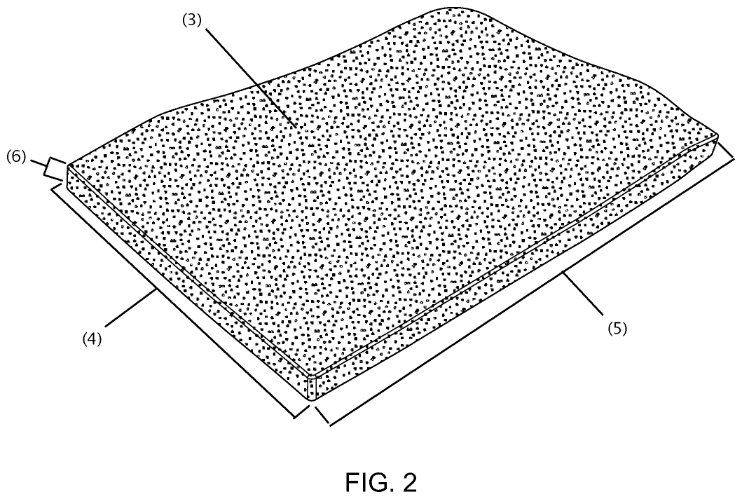

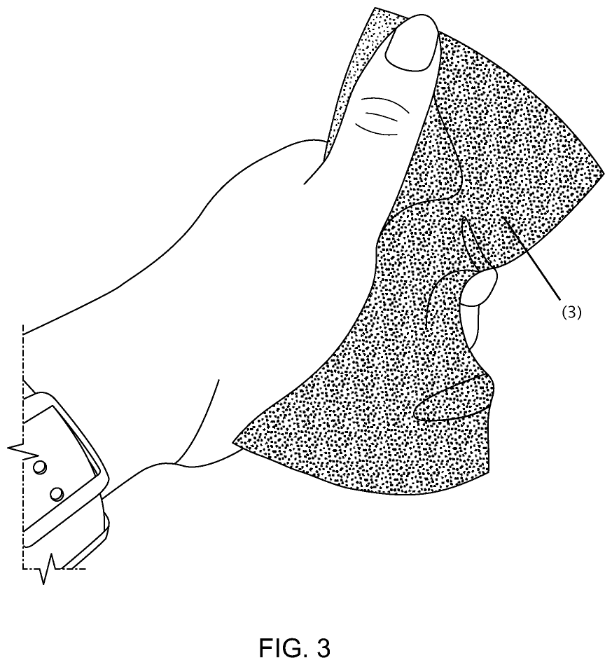

Reference is made to FIGS. 1-3, which employ a common numbering scheme.

Item (1) designates the body of the plastic covering while Item (2) designates a perforation in the plastic to assist in opening the plastic. It is this packaging the maintains the compression of the invention for transport.

Item (3) designates the body of the compressed sponge. Item (4) designates the lateral axis of invention. Item (5) designates the horizontal axis of invention. Item (6) designates the height axis of invention.

In a preferred embodiment, the lateral axis (4) is approximately two inches long. In a preferred embodiment, the horizontal axis (5) is approximately three inches long. In a preferred embodiment, the height axis (6) is approximately five millimeters (⅓ inch) in height when compressed. In a preferred embodiment, the height axis is approximately one inch when the sponge is expanded.

As used herein, the process of infusing the sponge with soap refers to adding and drying a liquid soap into the compressed sponge, such that the sponge is dry when packaged and transported, although contains dried liquid soap.

In a preferred embodiment, the sponge is made from polyether polyurethane.

In a preferred embodiment, the soap is made from the group consisting of Ammonium Laureth Sulfate, Ammonium Lauryl Sulfate, Sodium Laureth-40 Sulfate, Cocamidopropyl Betaine, glycerin, Cocamide DEA, Polyquaternium-7, Sodium chloride, Polydimethylsiloxane, Ethylene glycol stearate, Allantoin, Panthenol, Citric acid, DMDM hydantoin, Hypromellose, Methyl isothiazolinone, Magnesium Chloride, Magnesium Nitrate, and Fragrance.

In a preferred embodiment, the invention is disposed of after a single use once all the pre-packaged liquid soap is exhausted.

However, it is envisioned that the invention can be used in a daily routine, or reused if the liquid soap is not exhausted. At that point, the sponge portion of the invention can be reused as a sponge when the soap is exhausted.

It is also envisioned that the invention described herein can be utilized in other manner of applications. Non-limiting examples includes face sponges, dish sponges, and car sponges.

I claim:

1. A product comprising:
a compressed sponge;
a dried liquid soap;
wherein said compressed sponge and said dried liquid soap are packaged together as a single unit in a plastic wrapper; and
wherein the height of said compressed sponge expands by approximately three times when it is placed under a stream of water.

2. The product of claim 1 wherein the compressed sponge is formed by compressing a sponge of one inch in height.

3. The product of claim 1 wherein the compressed sponge expands to approximately one inch in height when it is placed under a stream of water.

4. The product of claim 1 wherein the dried liquid soap is dried onto said compressed sponge.

5. The product of claim 1 wherein the sponge is made from polyether polyurethane.

6. The product of claim 1 wherein the sponge is made from the group consisting of Ammonium Laureth Sulfate, Ammonium Lauryl Sulfate, Sodium Laureth-40 Sulfate, Cocamidopropyl Betaine, glycerin, Cocamide DEA, Polyquaternium-7, Sodium chloride, Polydimethylsiloxane, Ethylene glycol stearate, Allantoin, Panthenol, Citric acid, DMDM hydantoin, Hypromellose, Methyl isothiazolinone, Magnesium Chloride, Magnesium Nitrate, Fragrance.

7. The product of claim 1 wherein the sponge is utilized for personal human hygiene.

8. The product of claim 1 wherein the sponge is utilized to wash dishes.

9. The product of claim 1 wherein the sponge is utilized to wash cars.

10. A method of using a product, comprising:
compressing an uncompressed sponge to approximately five millimeters in height;
adding a bath soap to sponge to create a compressed sponge infused with soap;
packaging said compressed sponge infused with soap in a small plastic container; and
removing said compressed sponge infused with soap from said small plastic container and said compressed sponge infused with soap expands to the same height as said uncompressed sponge.

11. The product of claim 10 wherein the sponge is made from polyether polyurethane.

12. The product of claim 10 wherein the sponge is made from the group consisting of Ammonium Laureth Sulfate, Ammonium Lauryl Sulfate, Sodium Laureth-40 Sulfate, Cocamidopropyl Betaine, glycerin, Cocamide DEA, Polyquaternium-7, Sodium chloride, Polydimethylsiloxane, Ethylene glycol stearate, Allantoin, Panthenol, Citric acid, DMDM hydantoin, Hypromellose, Methyl isothiazolinone, Magnesium Chloride, Magnesium Nitrate, Fragrance.
